(19) United States
(12) Patent Application Publication
(10) Pub. No.: US 2018/0117359 A1
(43) Pub. Date: May 3, 2018

(54) PATIENT POSITIONING TRAINING APPARATUS

(71) Applicant: VISION RT LIMITED, London (GB)

(72) Inventor: Gideon HALE, London (GB)

(21) Appl. No.: 15/568,582

(22) PCT Filed: Apr. 22, 2016
(86) PCT No.: PCT/GB2016/051128

§ 371 Date: Oct. 23, 2017

(30) Foreign Application Priority Data
Apr. 24, 2015 (GB) 1507054.3

(51) Int. Cl. A61N 5/10; A61B 5/11

(52) U.S. Cl. CPC A61N 5/1049; A61B 5/1128; A61B 5/113; A61B 5/1114; A61N 5/1069; A61N 5/1037

(57) ABSTRACT

Some embodiments are directed to a method of assisting a patient in controlling their position, including obtaining a reference position data) indicative of a reference position of a patient, and actual position data is monitored and compared with the reference position data to determine a deviation, and the intensity and/or brightness and/or hue angle and/or flicker rate of emitted light which is detectable by the patient as ambient light is changed if there is a position deviation.

### CROSS REFERENCE TO RELATED APPLICATION(S)

This application is a national phase filing under 35 C.F.R. § 371 of and claims priority to PCT Patent Application No. PCT/GB2016/051128, filed on Apr. 22, 2016, which claims the priority benefit under 35 U.S.C. § 119 of British Patent Application No. 1507054.3, filed on Apr. 24, 2015, the contents of each of which are hereby incorporated in their entireties by reference.

### BACKGROUND

Some embodiments relate to patient positioning, and in particular to a method of helping a patient control their position. Some embodiments are especially suitable for use with radiotherapy devices and computed tomography (CT) scanners and the like, where controlling the patient's position is an important factor for successful treatment or diagnostic scanning.

Radiotherapy can include or can consist of projecting onto a predetermined region of a patient's body, a radiation beam so as to destroy or eliminate tumors existing therein. Such treatment is usually carried out periodically and repeatedly. At each medical intervention, the radiation source must or should be positioned with respect to the patient in order to irradiate the selected region with the highest possible accuracy to avoid radiating adjacent tissue on which radiation beams would be harmful.

It can therefore be important that the patient does not move during treatment. Particular problems arise where the tumors exist in the thoracic region of the patient as movement caused by breathing results in the movement of the tumor and the surrounding tissue.

It is known to monitor the position of a patient undergoing treatment. When applying radiation to a patient, the gating of a treatment apparatus can then be matched with a patient's position so that radiation is focused on the location of a tumor and collateral damage to other tissues is reduced or minimized. If movement of a patient is detected the treatment should be halted to avoid irradiating areas of a patient other than a tumor location.

Whilst it is possible to temporarily halt the treatment if excessive patient movement is detected, it is beneficial if the patient controls their own movement, particularly the movement associated with breathing so that it is more regular and excessive movement is reduced or minimized. If this occurs when a patient is in a CT scanner or the like, this can improve the quality of diagnostic images and hence improve the accuracy with which radiation can be targeted since the variation in location between different breathing cycles is reduced and blurring in diagnostic images is reduced. Better regulated breathing during treatment increases the likelihood that a tumor will follow the consistent path across different breathing cycles and hence increases the likelihood that a tumor will be located in an expected position when radiation is applied.

US2007093723 describes a radiotherapy device having a screen which shows a marker to provide feedback on a patient's breathing cycle between inhale and exhale limits. Whilst such a device does provide a visual representation of the patient's breathing cycle, the use of markers is not easy for the patient to follow in a high stress situation such as during radiotherapy treatment.

### SUMMARY

It may therefore be beneficial to enhance or improve devices for assisting a patient in controlling the patient's position.

Some embodiments are therefore directed to a method of assisting a patient in controlling the patient's position, including: defining reference position data indicative of a reference position of a patient monitoring the actual position to obtain actual position data, comparing the actual position data with the reference data to determine a position deviation, varying one or more of the intensity and/or brightness and/or hue angle and/or flicker rate of the emission of light detectable by the patient as ambient light if there is a position deviation.

Some other embodiments are directed to a position training apparatus including a determination module operable to determine actual position data indicative of the position of a patient, a signal comparison module operable to utilise the actual position data and identify a deviation between the actual position data and reference position data, and a light control module operable to vary the intensity and/or brightness and/or hue angle and/or flicker rate of the emission of light detectable by the patient as ambient light if there is a deviation between the actual position data and the reference position data.

Advantageously, light is emitted when the patient deviates from a reference position giving the patient the opportunity to move back into the correct position. The fact that the light is detected as ambient light is beneficial as it is less intrusive to the patient in what is already a high stress environment. Further the use of ambient light means that a patient does not need to view a light source directly and hence increases the flexibility of the location of the light source used to convey information to a patient. This increased flexibility can be of particular importance in the context of radiotherapy as frequently there is very little room available in a treatment room or within a scanning apparatus for additional equipment.

Furthermore, in contrast to using a screen with a marker which must or should firstly be positioned in the patient's line of sight which is not usually practical in a confined area, and secondly may require the patient to focus on a specific point, the light source can be positioned anywhere in a treatment room, including out of the line of sight of the patient, and may not require the patient to focus on the light source itself.

Preferably the reference position data is reference breathing data indicative of a reference breathing cycle of the patient, and the actual position data is actual breathing data indicative of the actual breathing cycle of the patient, the position deviation is a breathing deviation, further including controlling the intensity and/or brightness and/or hue angle and/or flicker rate of the emission of light detectable by the patient as ambient light with the reference breathing data, and changing one or more of the intensity and/or brightness and/or hue angle and/or flicker rate if there is a breathing deviation.

Advantageously, the emission of light gives a visual indication of the reference breathing cycle to the patient which can be followed, with any deviation from the reference being indicated by a change in the intensity and/or brightness and/or hue angle and/or flicker rate of the light which then gives the patient the opportunity to correct their breathing behavior.

Preferably, controlling the emitted light varies one or more of the intensity and/or brightness and/or hue angle and/or flicker rate.

Compared to known patient feedback systems using markers to visualise the breathing cycle, varying the emitted light is easier for the patient to follow, particularly in a confined and high stress environment, and therefore it is easier for the patient to control their breathing behavior.

Preferably, one or more of the intensity and/or brightness and/or hue angle and/or flicker rate increases with the reference breathing data. This is advantageous as the patient can associate increases of the intensity and/or brightness and/or hue angle and/or flicker rate with increases in the reference breathing data.

Preferably one or more of the intensity and/or brightness and/or hue angle and/or flicker rate varies in proportion to the position deviation which means the deviation can be visually detected by the patient as a change in the intensity and/or brightness and/or hue angle and/or flicker rate.

Alternatively, one or more of the intensity and/or brightness and/or hue angle and/or flicker rate is constant with the position deviation. This can be advantageous if for example the patient only wants to be alerted to the fact that there is a deviation, and not the extent of the deviation.

Examples of light properties include brightness, intensity, hue angle, and flicker rate. The properties can be chosen according to patient preference, for example a color blind patient might respond better to a change in intensity as opposed to a change in color, or a patient suffering from epilepsy would respond better to color changes than changing the flicker rate.

The breathing data can be breathing displacement and/or breathing cycle period. The intensity and/or brightness and/or hue angle and/or flicker rate of the emitted light can then be varied with the actual breathing data to visualise the actual breathing data to the patient.

The breathing deviations are defined by the difference between the actual breathing displacement and acceptable upper and lower breathing displacements, and/or the difference between the actual breathing cycle period and a reference period.

Preferably the color of the light changes when there is a deviation from the reference limits with a color change from green to blue being more possible. This benefits some patients who respond better to changing colors than say a change in intensity or brightness.

Preferably the light is a continuous light as this ensures immediate feedback to the patient when the light properties change as a result of the patient's position, and continuous visualization of the reference cycle which the patient is trying to follow.

Preferably the light detected by the patient is reflected light. This is particularly beneficial as the light appears more evenly distributed, and less intense, further enhancing the ambient nature of the light which is one of the many important factors in what is a high stress situation for the patient.

In an alternative embodiment, instead of conveying positional information to the patient using light, it is possible to use sound, and vary the sound in the same way, for example varying the tone and/or amplitude of the sound with reference positions and deviations from reference positions.

### BRIEF DESCRIPTION OF THE FIGURES

Some embodiments will now be described by way of example only with reference to the accompanying drawings in which:

FIG. 1 is a perspective view of a treatment system including a position training apparatus according to one aspect of the presently disclosed subject matter,

FIG. 2 is a schematic diagram of the position training apparatus of FIG. 1,

FIGS. 4 to 8 are examples of breathing signals generated by the breathing training apparatus of FIG. 1, and

FIG. 9 is a schematic end view of an alternative treatment system.

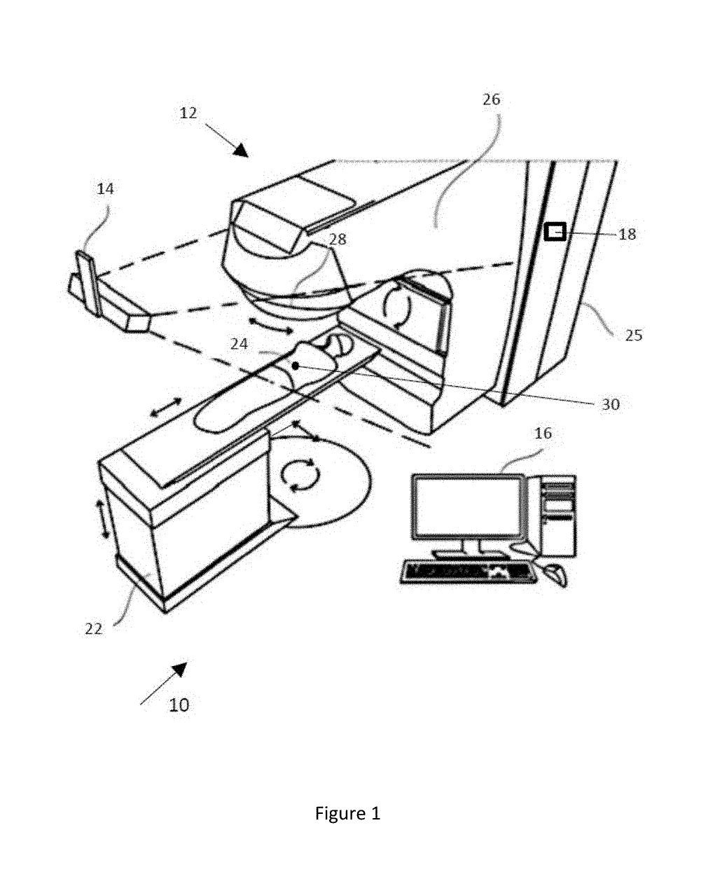

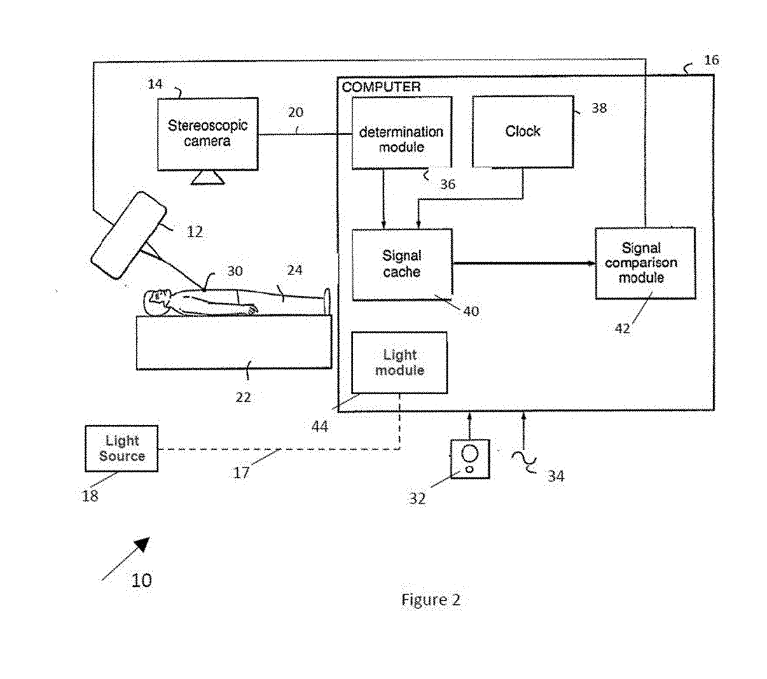

FIGS. 1 and 2 are a perspective view of a treatment system 10 including a position training apparatus and a schematic diagram of the position training apparatus.

### DETAILED DESCRIPTION

In FIG. 1, a treatment system 10 includes a treatment apparatus 12 such as a linear accelerator for applying radiotherapy or an x-ray simulator for planning radiotherapy, a stereoscopic camera 14, a computer 16 and a light source 18.

The stereoscopic camera 14 can be connected to the computer 16 either wirelessly or with a wire. In this embodiment, the stereoscopic camera 14 is connected to the computer 16 via a wire 20 (FIG. 2). The light source 18 is connected wirelessly (shown as a dashed line 17 in FIG. 2) to the computer 16. The computer 16 is also connected to treatment apparatus 12.

A mechanical couch 22 is provided upon which a patient 24 lies during treatment. The treatment apparatus 12 and the mechanical couch 22 are arranged such that under the control of the computer 16 the relative positions of the mechanical couch 22 and the treatment apparatus 12 may be varied, laterally, vertically, longitudinally and rotationally.

The treatment apparatus 12 includes a main body 25 from which extends a gantry 26. A collimator 28 is provided at the end of the gantry 26 remote from the main body of the treatment apparatus 12. To vary the angles at which radiation irradiates the patient 24, the gantry 26, under the control of the computer 16, is arranged to rotate about an axis passing through the centre of the main body of the treatment apparatus 12. Additionally the location of irradiation by the treatment apparatus may also be varied by rotating the collimator 28 at the end of the gantry 26.

The stereoscopic camera 14 obtains video images of the chest region 30 of the patient 24 lying on the mechanical couch 22. These video images are passed to the computer 16. The computer 16 then processes the images of the patient 24 to generate data representative of the patient's breathing cycle.

In this embodiment the light source 18 is positioned on the main body 25 of the treatment apparatus 12, and includes a plurality of individual light sources (not shown). The light sources can be any light source that is configurable to independently vary the properties of the emitted light, for example the hue angle, lightness, intensity, or flicker rate in response to a signal from the computer 16.

In terms of light properties, hue angle is used to describe the color of the light, so for example green and blue have different hue angles, brightness is also referred to as lightness which ranges between black and white, and intensity (or chromacity) is the strength of the color.

Typically, light emitting diodes (LEDs) would be suitable, where it will be appreciated that a combination of different colored LEDs may be required to be able to adjust some of the properties in combination with varying the current.

It can be seen from FIG. 1 in this example that the light source 18 is positioned on the side of the treatment apparatus 12 and out of the line of sight of the patient 24 such that, in use, the patient 24 does not see the emitted light from the light source 18 as a direct light, but rather perceives the light as background or ambient light.

Referring to FIG. 2, in order for the computer 16 to process the video images received from the stereoscopic camera 14 the computer 16 is configured by software either provided on a disk 32 or by receiving an electrical signal 34 via a communications network into a number of functional modules 36-44. It will be appreciated that the functional modules 36-44 shown in FIG. 2 are purely notional in order to assist with the understanding of the working of the claimed invention and may not in certain embodiments directly correspond with blocks of code in the source code for the software. In some embodiments the functions performed by the illustrated functional modules 36-44 may be divided between different modules or may be performed by the re-use of the same modules for different functions.

In this embodiment, the functional modules 36-44 include a determination module 36 for processing images received from the stereoscopic camera 14 and determining position data PD in the form of breathing data AT on the basis of a detected position for the patient, a clock module 38 for providing a timestamp for the breathing data determined by the determination module 36, a signal cache 40 for storing time stamped breathing data, a signal comparison module 42 to identify how the breathing data differs from reference breathing data, and a light control module 44 for controlling the emission of light.

Patient breathing data is obtained as follows:

Images are obtained by the stereoscopic camera 14, these images are processed by the determination module 36 which determines the position of the chest 30 of the patient. The determination module 36 then converts this position signal into a distance measure indicating the relative distance of the chest 30 from a fixed reference point. This distance data is then stored in the signal cache 40 together with timing data indicating the timing at which the image of the chest 30 was obtained by the computer 16 to generate actual breathing data AT which is indicative of the patient's breathing cycle.

It will be appreciated that by obtaining images of the patient over time T, actual breathing data in the form an actual breathing trace AT can be generated.

The actual breathing trace AT can then be compared with a reference breathing trace RT corresponding to a normal or desired breathing cycle of the patient to determine if there is a deviation.

During the breathing cycle, the patient 24 will repeatedly inhale and exhale. FIG. 3 shows a sample breathing trace BT which includes an inhale portion (position E to position F) and an exhale portion (position F to position G). When the patient inhales, the breathing displacement BD increases with time until it reaches a maximum positive breathing displacement (F) before exhaling to reach a maximum negative breathing displacement (position G). The breathing displacement (BD) and the period P which is the time between successive positive or negative maximum breathing displacements can be compared with corresponding reference values as will be described below.

The patient 24 is assisted in regulating their breathing as follows:

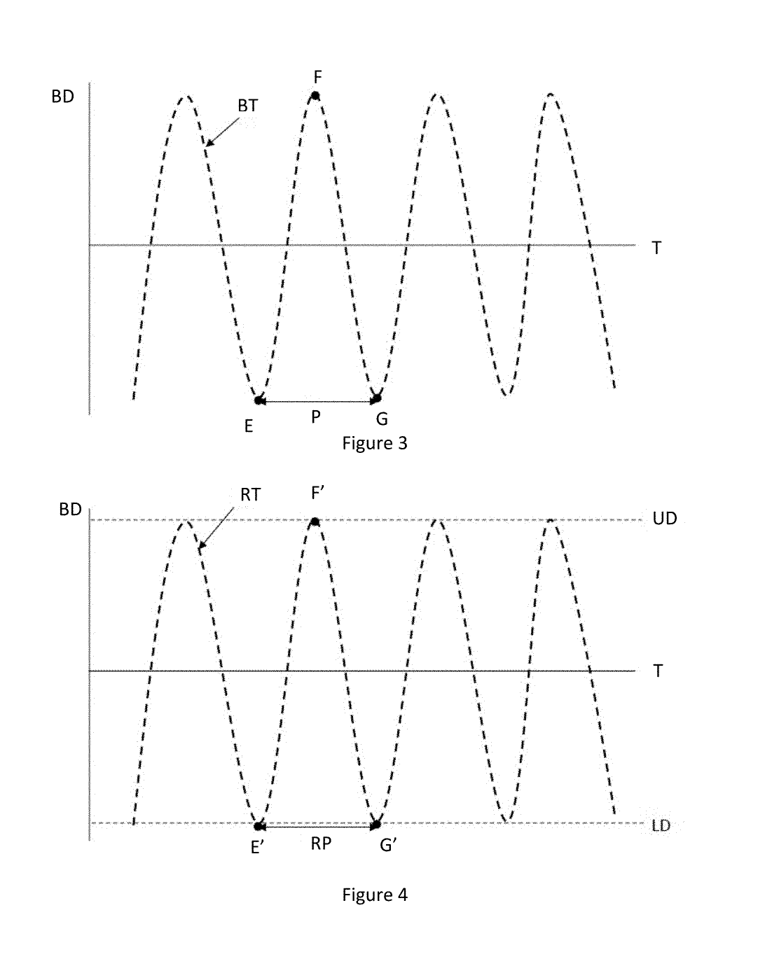

With reference to FIG. 4, firstly a reference breathing trace RT is chosen for a particular patient. The reference trace RT can be a previously obtained reference trace for the patient, or a reference trace chosen which corresponds to the breathing cycle most appropriate for the treatment. The breathing reference trace indicates reference breathing data in the form of an upper displacement limit UD and a lower displacement limit LD, and reference period RP.

When the patient 24 is in position prior to treatment, the light control module 44 sends a signal to the light source 18 to emit a continuous (i.e. no flicker) light with a first color, in this embodiment, green light L with a fixed brightness and an intensity that varies in proportion to the reference breathing trace RT, i.e. the intensity increases with the inhale portion (E′ to F′) of the reference trace RT, and decreases with the exhale portion (E′ to G′) of the reference trace RT (FIG. 4). The patient 24 will detect the light as ambient light whilst undergoing treatment and attempt to regulate their breathing to following the varying intensity of the light, and therefore the reference breathing trace RT corresponding to a normal breathing cycle. In particular, the patient 24 will try to regulate the point at which they stop inhaling and begin exhaling (F′) which is detectable as the intensity stops increasing and starts decreasing, and stop exhaling/begin inhaling (G′) which is detectable as intensity stops decreasing and starts increasing.

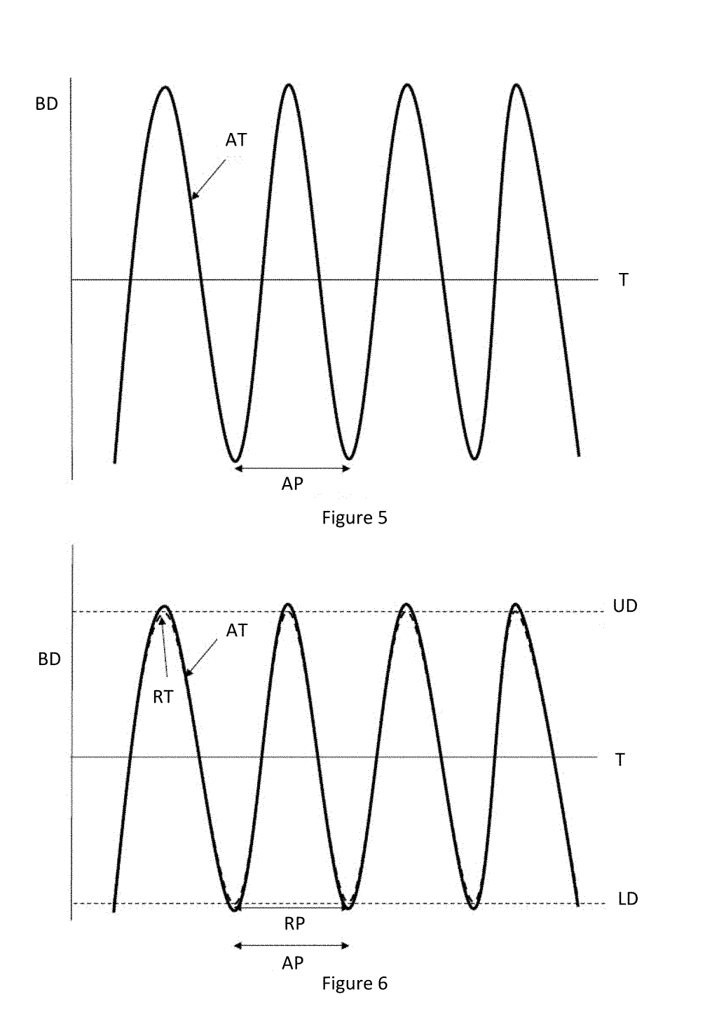

At the same time as the patient 24 is attempting to regulate their breathing by following the reference trace RT, the actual breathing trace AT is obtained as described above (FIG. 5). The actual breathing trace AT is compared to the reference breathing trace RT using the signal comparison module 42. In this embodiment, the actual breathing displacement BD is compared to the upper displacement limit UD and the lower displacement limit LD.

If the breathing displacement BD of the actual trace AT remains within the upper and lower LD displacement limits (as shown exaggerated for illustrative purposes in FIG. 6) the light control module 44 continues to send a signal to the light source 18 to emit green light with an intensity that varies in proportion to the reference breathing trace RT, i.e. the patient sees no change in the ambient light and will continue to attempt to follow the reference breathing trace RT in the knowledge that the actual breathing displacement is within the upper UD and lower LD displacement limits.

If the breathing displacement BD of the actual trace AT is outside the upper or lower LD displacement limits, the light control module 44 sends a signal to the light source 18 to change the color of the light to a second color, in this embodiment, blue, i.e. the patient sees a step change in the color of the ambient light from green to blue, and will recognize that the actual breathing displacement has gone outside the upper displacement UD or lower displacement LD limits, and will attempt to regulate the breathing by inhaling or exhaling less. When the patient regulates their breathing to the extent that the displacement returns inside the upper UD or lower LD displacement limits, the light control module 44 sends a signal to change the color back to green again and the patient will again attempt to follow the reference breathing trace RT.

If the breathing displacement goes outside the upper UD or lower LD displacement limits, the intensity of the blue light continues to vary with the actual breathing trace AT, i.e. the light gets more intense as the breathing displacement BD deviates further above the upper displacement limit UD and less intense as it returns towards the upper displacement limit displacement UD, and less intense as the breathing displacement BD deviates further below the lower displacement limit LD, and more intense as it returns towards the lower displacement limit LD.

In another embodiment, the intensity of the light can remain constant if the breathing displacement BD is outside of the upper UD or lower LD displacement limits such that the patient simply sees a color change that is not dependent on the deviation from the upper UD or lower LD displacement limits.

In another embodiment, other properties of the light can vary instead of the intensity, for example, the brightness, hue angle or flicker rate can all or mostly be used either individually, or in different combinations to vary with the reference RT or actual AT breathing traces, the choice being dependent on patient preference.

For the same reasons, other properties of the light can be changed instead of changing the color from green to blue to indicate the actual breathing displacement BD has gone outside limits, for example, the intensity, brightness, hue angle or flicker rate can all or mostly change either individually, or in different combinations. Furthermore, those same properties can be varied in proportion to the breathing deviation from the upper or lower limits.

It will be appreciated that the first and second colors need not be limited to green and blue. To indicate a deviation from the reference breathing trace RT, a step change in color that is clearly apparent to the patient may be required, and this typically may require change of color from one part of the color spectrum to another. The first color can therefore be selected from one of the blue, green, red, violet, orange or yellow spectrums, and the second color also selected from one of the blue, green, red, violet, orange or yellow spectrums but different from the first color. Patient preference again dictates the selection of the first and second colors.

If the hue angle is chosen to represent the variation of the reference breathing trace RT, then any step change in color to represent a deviation from the reference breathing trace RT needs to be clearly distinguishable from the variation in hue angle in order for the patient to recognize the color change. For example, if the hue angle varies from green to blue with the reference breathing trace RT, any deviation can be indicated by changing the color to red. The patient will therefore clearly recognize the change to red, and understand the actual breathing displacement BD has gone outside the upper UD or lower LD displacement limits.

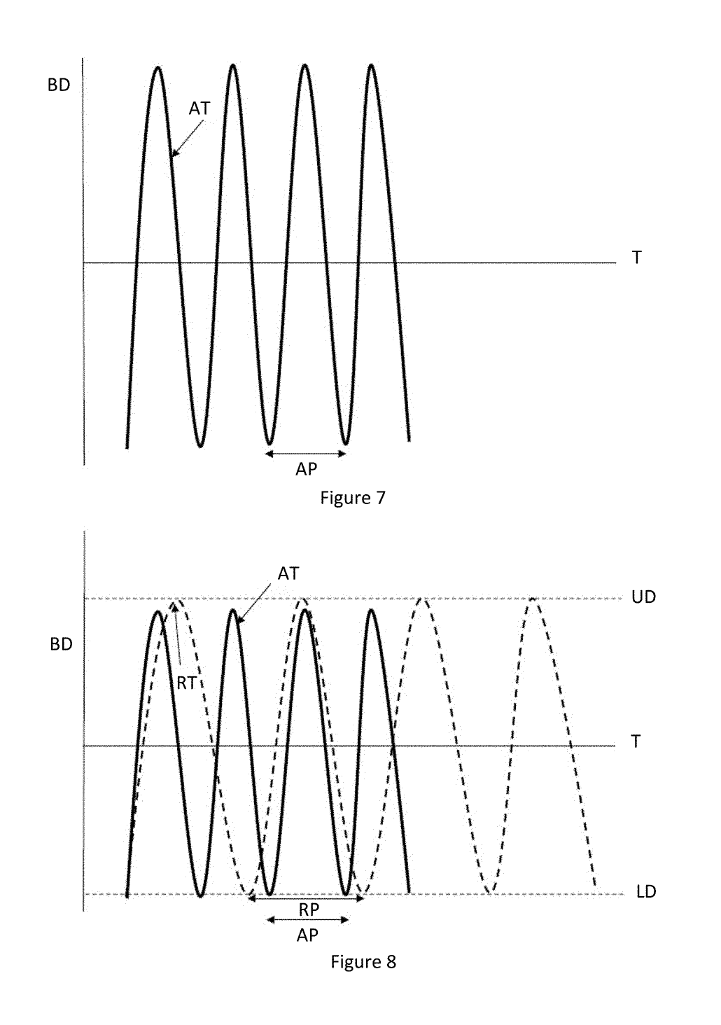

In another embodiment, the actual period AP to complete a breathing cycle can be compared to a reference period RP (FIG. 7). The patient still follows the green light corresponding to the reference breathing trace RT as described above, however if the actual period AP deviates from the reference period RP (FIG. 8), the light control module 44 sends a signal to change the color of the light to a second color, in this case blue. It is possible to configure the light control module 44 so that the color changes whenever the actual period AP deviates from the reference period RP, or if the actual period AP is greater or less than the reference period RP depending on the requirements. For example, it may be that the patient typically breaths at too fast a rate (the period is too low) in which case a visual indication of the actual breathing period AP being below the reference period RP is useful to remind the patient he is breathing too fast.

It will be appreciated that both actual the breathing displacement BD and the period AP can simultaneously be compared to reference values, and the light control module 44 can be configured to send a signal to indicate if either the breathing displacement BD, and/or the period deviates from the reference values depending on the requirement. For example, in some situations it may be necessary to regulate the breathing displacement to within certain limits to reduce or minimize patient movement. In other situations, a regular breathing cycle period is considered to be more one of the many important so that the maximum positive and negative displacements, and hence the time at which the treatment is applied, is more regular.

To avoid confusing the patient, different colors can be used to indicate whether the deviation relates to the breathing displacement or the period. This may be important in situations that may require feedback of both the breathing displacement and the period to the patient.

Instead of using different colors to distinguish between breathing displacement and period deviations it is possible to vary other light properties. For example, deviations of breathing displacement can be indicated by a change in color, whereas deviations from the reference period can be indicated by a change in the brightness. As has already been described above, selection of light properties is very much dependent on patient preference as it is critical that the patient is not startled by the light, and responds to any changes so as to follow and modify breathing behavior.

The light emitted in some embodiments above is emitted as a continuous light, by which it is meant the light continues to be emitted as the light properties such as intensity, hue angle, or brightness are changed. It is also possible to interrupt the light by introducing flicker, with the flicker rate determined by the actual breathing data or deviations from the reference data. For example, the light could be emitted with the intensity changing with the reference breathing trace, and a constant flicker rate. Any deviations from the reference breathing trace, for example an increase in the breathing frequency (corresponding to a shorter period as a result of more rapid breathing) could be indicated by the flicker rate increasing, the reverse being the case if the breathing frequency is too low.

In some embodiments described above, the light property, be that brightness, intensity, hue angle, or flicker rate increases with increasing breathing displacement. In an embodiment the light property could decrease, so for example the intensity could decrease as the breathing displacement increases. Similarly, deviation from reference breathing displacement could result in the light property decreasing as opposed to increasing as described in some of the embodiments above. As described above, the variation in light is very much chosen according to patient preference.

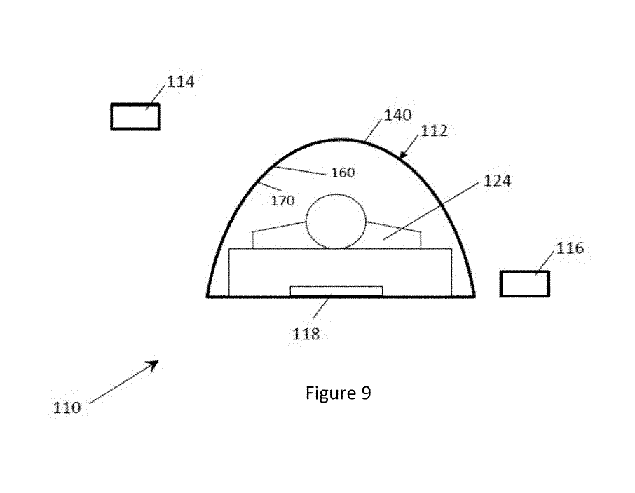

FIG. 9 shows an alternative treatment system 110 which is identical to the treatment system of FIG. 1 apart from the treatment apparatus.

The treatment system 110 includes a treatment apparatus 112, a stereoscopic camera 114, a computer 116, and a light source 118.

The treatment apparatus 112 is typical of radiation planning apparatus such as a CT scanner which have a small part-cylindrical tube-like portion 140 that is not much greater in internal diameter than the patient in which it houses.

The tube-like portion 140 has an inside surface 160 to which is applied a reflective or light colored coating 170. The light source 118 is positioned at one end of the tube-like portion 140 out of the line of sight of the patient 124. It can be seen from FIG. 9 that there is very little space inside the tube-like portion 140, and therefore little room to position the light source 118.

In use, the light source 118 operates in the same way as the light source of FIG. 1 under the control of the light control module (not shown). The emitted light reflects off of the reflective or light colored coating 170 to further distribute the light evenly around the tube-like portion 140 to give the light an even greater ambient appearance. The tube-like portion 140 is a cramped environment and therefore it is advantageous to be able to position the light source 115 with some freedom. In addition, relying on reflected light prevents the patient from being startled as the light has a more ambient appearance. This is particularly important in what is a high stress environment for the patient.

In the treatments systems of FIGS. 1 and 9 the light source 18,118 is positioned on the treatment apparatus. In an alternative treatment system the light source can be remote from the treatment apparatus, for example on a wall or ceiling or floor of a treatment room. The light source in the treatment room can be similarly arranged such that it is out of the line of sight of the patient. Furthermore, a reflective coating can be applied to the walls, ceiling and floor to reflect light more evenly from the light source around the room to give the light an even greater ambient appearance when detected by the patient.

The embodiments above describe varying light properties which are detected by the patient to help regulate breathing. Some embodiments have been described where the emitted light varies according to reference breathing data to help the patient following the reference, and where the light varies again to indicate any deviations from the reference breathing data, be that the breathing displacement and/or the breathing period. The light properties can be kept constant or varied depending on how the actual breathing data compares to the reference breathing data, and according to patient preference, as different patients respond to different visual stimuli. The light properties can be varied by varying the brightness, lightness, hue angle (color) or the flicker rate, again according to patient preference. It will be appreciated that key to some embodiments is the ability to emit light with changing properties that helps a patient regulate their breathing cycle, and then change the properties of the light to indicate if the breathing cycle deviates from the reference cycle. Another key requirement is that the patient is not startled by the emission of that light, and for this reason the light source is arranged relative to the patient such that the light is detected by the patient as ambient light. The fact that the light is ambient light is particularly advantageous as it enables the light source to be positioned inside treatment apparatus with restricted space, or even utilising a light source that is remote from the apparatus.

Some embodiments above describe a light source whose output is varied by controlling the light source itself, for example using a combination of different colored LEDs and/or varying the current to different LEDs to emit light of varying properties (hue, intensity, brightness).

Whilst the light detectable by the patient appears ambient in nature, the properties of the light are controlled by the light source. In an alternative embodiment, an optical feature such as a filter, or a combination of filters can be provided between the light source and the patient so as to change the properties of the light after is has been emitted by the light source. Furthermore, the optical feature can be controlled so as to vary the properties of the emitted light in response to the actual and reference data in the same way as the light source itself is varied.

The above embodiments monitor the patient's position, that position being exemplified by breathing behavior, and give feedback to the patient to help control their breathing behavior. This is particularly important when the tumors exist in the thoracic region of the patient as movement caused by their breathing results in the movement of the tumor and the surrounding tissue. For treatments in other locations, whilst helping the patient control their breathing is important, it can be as, if not more important to limit other forms of movement of the patient not associated with breathing, for example it could be important for another part of the body being treated to be kept still, with any detected movement conveyed to the patient with the emission of light as discussed above. In the same way as breathing deviations results in changes in the light properties, the same changes be applied to any other patient movement. For example, a light of a particular color, for example green, could be emitted when part of the patient's body is in the correct (reference) position, with a change in color of the light to red if the body part deviates from that position. Again, in the same way as with breathing deviations, the extent of the deviation could be conveyed by varying the light properties such as the intensity, brightness or flicker rate, or indeed any combination of those light properties.

It will be appreciated that the patient's body has six degrees of freedom, and therefore the monitoring of the patient needs to able to detect deviation in any direction. Furthermore, the light can varied to convey particular information to the patient depending on which direction they are moving. For example, if the patient's hand is being treated, a green light could indicate vertical deviations, and a red light, horizontal deviations. The possibilities are numerous, and as has been emphasised in relation to varying the light in relation to breathing behavior, patient preference is key to ensuring the light properties are changed in such a way that will enable the patient to respond.

In some embodiments, light, and varying the properties of light, is used to convey positioning information, and deviations from reference positions to the patient. Not every patient responds well to light changes, and indeed for visibly impaired patients the use of light is not appropriate. Instead of using light to convey information to the patient, it is possible to use sound, and in the same way as the light is varied by changing color, brightness, intensity and flicker rate, the sound can be varied by changing the amplitude (volume), and/or the tone. In some embodiments, a combination of light and sound could be used. For example, the emitted sound could indicate the reference positioning data, for example the breathing cycle, with any deviation from the reference resulting in light being emitted. Again the possibilities to vary the sound alone, or in combination with the light are numerous, and depend on patient preference.

It will be appreciated that a sound control module (not shown) may be required to control the emitted sound, and this is connected to the computer either by a physical wired connection or a wireless connection.

1. A method of assisting a patient in controlling their the patient's position, comprising the steps of:
defining reference position data indicative of a reference position of the patient;
monitoring an actual position of the patient to obtain actual position data;
comparing the actual position data with the reference data to determine a position deviation; and
varying at least one of intensity and/or brightness and/or hue angle and/or flicker rate of light emission detectable by the patient as ambient light upon determination of the position deviation.

2. The method of claim 1, wherein which the reference position is reference breathing data indicative of a reference breathing cycle of the patient, and the actual position data is actual breathing data indicative of the actual breathing cycle of the patient, the position deviation is a breathing deviation, the method further including:
controlling at least one of the intensity and/or brightness and/or hue angle and/or flicker rate of the light emission detectable by the patient as ambient light with the reference breathing data; and
changing at least one of the intensity and/or brightness and/or hue angle and/or flicker rate upon detection of a breathing deviation.

3. The method of claim 2, wherein controlling the emission of light varies at least one of the intensity and/or brightness and/or hue angle and/or flicker rate.

4. The method of claim 3, wherein at least one of the intensity and/or brightness and/or hue angle and/or flicker rate increases with the reference breathing data.

5. The method of claim 1, wherein at least one of the intensity and/or brightness and/or hue angle and/or flicker rate varies in proportion to the position deviation.

6. The method of claim 1, wherein at least one of the intensity and/or brightness and/or hue angle and/or flicker rate is constant with the position deviation.

7. The method of claim 1, wherein reference and actual position data is positional displacement.

8. The method of claim 7, wherein the intensity varies in proportion to the positional displacement.

9. The method of claim 7, wherein the positional displacement is breathing displacement.

10. The method of claim 9, wherein the reference breathing displacement is an upper displacement limit and a lower displacement limit, the breathing deviation is the difference between the actual breathing displacement and the upper or lower displacement limits.

11. The method of claim 2, wherein the actual breathing data is an actual breathing period, and the reference breathing data is a reference breathing period, the breathing deviation is the difference between the actual breathing period and the reference breathing period.

12. The method of claim 2, wherein the color of the emitted light changes from a first color to a second color upon detection of a breathing deviation.

13. The method of claim 10 wherein the color of the emitted light changes from a first color to a second color when there is a breathing deviation, and the second color associated with a period breathing deviation differs from the second color associated with a displacement breathing deviation.

14. The method of claim 12, wherein the first color is in the green spectrum, and the second color is in the blue spectrum.

15. The method of claim 1, wherein the light is a continuous light.

16. The method of claim 1, wherein the light detected by the patient is reflected light.

17. (canceled)

18. (canceled)

19. (canceled)

20. A method of assisting a patient in controlling the patient's position, comprising:
defining reference position data indicative of a reference position of the patient;
monitoring an actual position to obtain actual position data;
comparing the actual position data with the reference data to determine a position deviation; and
emitting sound having at least one sound property upon determination of the position deviation.

21. The method of claim 20, wherein the reference position data is reference breathing data indicative of a reference breathing cycle of the patient, and the actual position data is actual breathing data indicative of the actual breathing cycle of the patient, the position deviation is a breathing deviation, the method further comprising:
emitting sound having at least one sound property, at least one of the at least one sound properties varying with the reference breathing data; and
changing at least one of the at least one sound properties upon detection of a breathing deviation.

22. The method of claim 2h wherein at least one of the at least one sound properties increases with the reference breathing data (RT).

23. The method of claim 21, wherein at least one of the at the least one sound properties varies in proportion to the position deviation.

24. A position training apparatus comprising:
a determination module configured to determine actual position data indicative of a patient's position;
a signal comparison module configured to utilize the actual position data, and identify a deviation between the actual position data and reference position data; and
a light control module configured to vary the intensity and/or brightness and/or hue angle and/or flicker rate of light emission detectable by the patient as ambient light and/or a sound control module operable to emit sound having at least one sound property upon identification of the deviation between the actual position data and the reference position data.

25. The position training apparatus of claim 24, wherein the reference position data is reference breathing data indicative of a reference breathing cycle of the patient, and the actual position data is actual breathing data indicative of the actual breathing cycle of the patient, the apparatus further comprising:
a clock module operable to associate the actual breathing data determined by the determination module with timing data; and
wherein the light control module is configured to control the intensity and/or brightness and/or hue angle and/or flicker rate of the emission of light detectable by the patient as ambient light with the reference breathing data, and change at least one of the intensity and/or brightness and/or hue angle and/or flicker rate if there is a breathing deviation, the sound control module is operable to emit sound having at least one sound property, one or more of the at least one sound properties varying with the reference breathing data, and change one or more of the at least one sound properties upon determination of a breathing deviation.

26. The position training apparatus of claim 24, further comprising at least one optical feature, the light control module being operable to control the at least one optical feature to vary the intensity and/or brightness and/or hue angle and/or flicker rate.

27. A treatment system, comprising:
a treatment apparatus for treating a patient; and
the position training apparatus according to claim 24.

28. The treatment system of claim 27, wherein the light source is positioned out of the line of sight of a patient undergoing treatment.

29. The treatment system of claim 27, wherein which the at least one light source is positioned on the treatment apparatus.

30. The treatment system of claim 27, wherein the at least one light source is remote from the treatment apparatus.

31. The treatment system of claim 27, wherein the treatment apparatus includes an interior surface, which reflects the light from the at least one light source towards the patient.

32. (canceled)

33. (canceled)

34. (canceled)
